(19) United States
(12) Patent Application Publication
(10) Pub. No.: US 2014/0308254 A1
(43) Pub. Date: Oct. 16, 2014

(54) METHODS AND COMPOSITIONS FOR TREATING DIABETES AND OTHER DEGENERATIVE NEUROENDOCRINE DISEASES OR DISORDERS

(71) Applicant: University of Louisville Research Foundation, Inc., Louisville, KY (US)

(72) Inventors: Fred J. Roisen, Prospect, KY (US); Chengliang Lu, Louisville, KY (US)

(21) Appl. No.: 14/354,004

(22) PCT Filed: Oct. 26, 2012
(86) PCT No.: PCT/US12/62113

§ 371 Date: Apr. 24, 2014

Related U.S. Application Data
(60) Provisional application No. 61/551,681, filed on Oct. 26, 2011.

(51) Int. Cl. A61K 35/30

(52) U.S. Cl. CPC A61K 35/30
USPC 424/93.7

(57) ABSTRACT

The present application describes the use of adult human olfactory neuroepithelium (ONe) stem cells in the treatment of diabetes.

### CROSS REFERENCE TO RELATED APPLICATIONS

This application claims benefit of priority to U.S. Application No. 61/551,681 filed Oct. 26, 2011.

### TECHNICAL FIELD

This disclosure generally relates to stem cells and their use in treating diabetes.

### BACKGROUND

Type I diabetes is usually diagnosed in children and young adults, and was previously known as juvenile diabetes. In type 1 diabetes, the body does not produce insulin or produces very little insulin. None of the cell therapies to date have been effective for treating diabetes in humans. A cell therapy for treating diabetes is described herein that avoids at least some of the problems reported with previous cell therapies.

### SUMMARY

It is shown herein that adult human olfactory neuroepithelium (ONe) stem cells produce insulin. Therefore, the adult human ONe stem cells can be used in a cell-based therapy for the treatment of diabetes or other degenerative neuroendocrine diseases.

In one aspect, a method of treating a patient having diabetes is provided. Such a method typically includes culturing human olfactory neuroepithelium stem cells ex vivo under appropriate conditions; and engrafting the human olfactory neuroepithelium stem cells into the patient. Generally, the human olfactory neuroepithelium stem cells produce insulin, thereby treating the patient having diabetes.

In another aspect, a method of treating a patient having diabetes is provided. Typically, such a method includes providing human olfactory neuroepithelium stem cells that have been cultured under appropriate conditions ex vivo; and engrafting the cultured human olfactory neuroepithelium stem cells into the patient. Generally, the human olfactory neuroepithelium stem cells produce insulin, thereby treating the patient having diabetes.

In still another aspect, a method of treating a patient having diabetes is provided. Typically, such a method includes harvesting human olfactory neuroepithelium stem cells from a patient having diabetes; culturing the human olfactory neuroepithelium stem cells ex vivo under appropriate conditions; and engrafting the human olfactory neuroepithelium stem cells into the patient. Generally, the human olfactory neuroepithelium stem cells produce insulin, thereby treating the patient having diabetes.

In some embodiments, the human olfactory neuroepithelium stem cells are autologous to the patient. In some embodiments, the human olfactory neuroepithelium stem cells are allogeneic to the patient.

In some embodiments, the harvesting step is performed using an endoscope. In some embodiments, the culturing step increases the number of human olfactory neuroepithelium stem cells. In some embodiments, the engrafting step comprises injecting the human olfactory neuroepithelium stem cells into the pancreas. In certain embodiments, the culturing step does not require additional growth factors. In certain embodiments, the culturing step comprises changing or adjusting the amount of glucose in the medium.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DETAILED DESCRIPTION

It is demonstrated herein that adult human olfactory neuroepithelium (ONe) stem cells produce insulin in a glucose-dependent manner. Therefore, such adult human ONe stem cells can be used in a cell-based therapy for the treatment of diabetes. Based on the results presented herein, adult human ONe stem cells are ideal for replacing or supplementing beta cells in the pancreas of an individual that has diabetes.

### The Adult Human ONe Stem Cells

The adult human ONe stem cells referred to herein are described in more detail in WO 2003/064601 and in Roisen et al. (2001, “Adult human olfactory stem cells,” Brain Res., 890:11-22). Adult human ONe stem cells are pluripotent and can differentiate into sensory and motor neurons, oligodendrocytes, and supporting glial cells. Adult human ONe stem cells are neural progenitors that have an apparently unlimited capacity for self-renewal and can form spontaneously contractile “cardiac-like” myocytes. They are angiogenic when engrafted into normal host tissue. The genetic potential of adult human ONe stem cells has been characterized (see, for example, Khalyfa et al., 2007, “Gene expression profiling for adult human olfactory neuroepithelial-derived progenitors,” Gene Ther. Mol. Biol., 11:203-16). Furthermore, these cells are characterized by the expression of nestin as well as the expression of beta-tubulin isotype III, NCAM, A2B5, MAP2 and peripherin. See, for example, Zhang et al. (2004, “Adult human olfactory neural progenitors cultured in defined medium,” Exp. Neurol., 186:112-23).

The adult human ONe stem cells referred to herein can be obtained from a live donor using a minimally invasive procedure such as endoscopy. See, for example, Winstead et al. (2005, “Endoscopic Biopsy of Human Olfactory Epithelium as a Source of Progenitor Cells,” Am. J. Rhinol., 19:83-90). As described therein, adult human ONe stem cells can be obtained from a biopsy of the olfactory area (e.g., superior turbinate, middle turbinate, dorso-posterior nasal septum) during a procedure similar to endoscopic sinus surgery. In some embodiments, the ONe stem cells are endogenous to the patient (i.e., obtained from the patient, cultured ex vivo, and engrafted back into the patient). In some embodiment, the ONe stem cells are allogeneic to the patient (i.e., obtained from a donor individual from the same species as the patient, cultured ex vivo, and engrafted into the patient).

The adult human ONe stem cells can be cultured using routine methods. For example, adult human ONe stem cells can be cultured in medium containing DMEM (Dulbecco's Modified Eagle Medium) and F12 (1:1) with 10% heat-inactivated fetal bovine serum (FBS). After culturing for several weeks, a population of mitotically active cells emerge while other cells present in the tissue (e.g., olfactory receptor neurons (ORNs), olfactory ensheathment or sustentacular cells (OECs) become vacuolated, retract their processes, and die after approximately three weeks in culture. The mitotically active cells, which are the adult human ONe stem cells, grow in suspension, double every day and, after two to three weeks of proliferation, neurospheres begin to form. As used herein, neurospheres refer to a cluster of about 20-80 mitotically active adult human ONe stem cells. Generally, neurospheres are a population of cells that, in addition to adult human ONe stem cells, include cells in various stages of differentiation. Neurospheres typically form spherical, tightly packed cellular structures.

The neurospheres can be collected, washed, and, if desired, dispersed into individual cells. The neurospheres or the individual cells can be probed with one or more antibodies to determine whether or not the cells express a particular protein marker. Such protein markers include, without limitation, nestin, beta-tubulin isotype III, NCAM, GFAP, Trk A, Trk B, peripherin, A2B5, MAP2, BDNF, NGF, CTNF, and NT-3. See, for example, Xiao et al. (2005, “Human adult olfactory neural progenitors rescue axotomized rodent rubrospinal neurons and promote functional recovery,” Exp. Neurol., 194:12-30) and Wang et al. (2011, “Lineage restriction of adult human olfactory-derived progenitors to dopaminergic neurons,” Stem Cell Discovery, 1 (3):29-43). The adult human ONe stem cells can be differentiated into a particular type of daughter cell; a small number of the adult human ONe stem cells can differentiate spontaneously, or exogenous factors such as retinoic acid, forskolin, and/or sonic hedgehog can be added to the medium to direct the cells down a particular lineage (see, for example, Zhang et al., 2006, “Induction of neuronal differentiation of adult olfactory neuroepithelial-derived progenitors,” Brain Res., 1073-4:109-19).

### Methods of Treating Diabetes

As demonstrated herein, adult human ONe stem cells produce insulin. Therefore, the adult human ONe stem cells can be engrafted into an individual that has type I diabetes. In addition, adult human ONe stem cells can be engrafted into an individual that has a different degenerative neuroendocrine disease or disorder including, but not limited to, pituitary disorders or tumors as well as disorders of the hypothalamus. An example of a degenerative neuroendocrine disease or disorder other than diabetes is Cushing's disease.

Engraftment of the adult human ONe stem cells can be achieved by direct injection into, for example, the pancreas or the capsule of the kidney. Alternatively, engraftment can occur via surgical implantation or other means.

There are recent reports of neural progenitors from the olfactory bulb or the hippocampus producing insulin and, thus, their potential application in diabetes is proposed. See Kuwabara et al. (2011, “Insulin biosynthesis in neuronal progenitors derived from adult hippocampus and the olfactory bulb,” EMBO Mol. Med., 3:1-13) and Basak & Clevers (2011, “Neural stem cells for diabetes cell-based therapy,” EMBO Mol. Med., 3:1-3). However, harvesting tissue or cells from the hippocampus or the olfactory bulb requires highly invasive surgery, and certainly cannot be removed without substantial risk to the patient.

Methods of “treating” diabetes refer to cell therapies that increase the amount of insulin produced by the patient. As used herein, “treating” diabetes can result in a reduction or an amelioration of one or more of the symptoms associated with diabetes (e.g., frequent urination, excessive thirst, hunger, unusual weight loss, fatigue and/or irritability).

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### The Adult Human ONe Stem Cells

Over 150-patient specific hONP lines were isolated from females and male with an age range of 28-87 years (Marshall et al., 2006, “The therapeutic potential of human olfactory-derived stem cells,” Hist. and Histopath., 21:633-43). There was no change in stability, telomerase, polyamine biosynthetic, or apoptotic activities, even during several years in culture (see, for example, Marshall et al., 2005, “Human adult olfactory neuroepithelial derived progenitors retain telomerase activity and lack apoptotic activity,” Brain Res., 1045:45-56). In addition, adult human ONe stem cells can be frozen in liquid nitrogen and maintained indefinitely for future use. See, for example, Zhang et al. (2004, “Adult human olfactory neural progenitors cultured in defined medium,” Exp. Neurol., 186:112-23); Marshall et al. (2006, “The therapeutic potential of human olfactory-derived stem cells,” Hist. Histopath., 21:633-43); Lu et al. (2011, “Human olfactory-derived neural progenitors diminish locomotory deficits following spinal cord contusion injury,” J. Neurodegen. & Regen., 3 (1):33-50).

In addition, adult human ONe stem cells can be clonally expanded without limitation (see, for example, Othman et al., 2005, “Adult human olfactory neurosphere forming cells: clonal analysis,” Biotechnic & Histochem., 80:189-200), and they continue to divide in the absence of exogenous growth factors (see, for example, Zhang et al., 2004, “Adult human olfactory neural progenitors cultured in defined medium,” Exp. Neurol., 186:112-23; and Marshall et al., 2006, “The therapeutic potential of human olfactory-derived stem cells,” Hist. & Histopath., 21:633-43). Therefore, adult human ONe stem cells obtained from an individual can be expanded to very large numbers and stored (i.e., frozen) for future or repeated use (see, for example, Othman et al., 2005, “Immunomagnetic separation of adult human olfactory neural progenitors,” Biotechnic & Histochem., 80:177-88; Lu et al., 2011, “Human olfactory-derived neural progenitors diminish locomotory deficits following spinal cord contusion injury,” J. Neurodegen. & Regen., 3 (1):33-50.

### Example 2

### Insulin Production by Adult Human ONe Stem Cells

Immunofluorescence studies using two different monoclonal antibodies to insulin were performed. The anti-insulin antibodies used were monoclonal anti-insulin antibodies (e.g., Catalog #ab9569 (E2E3) (abcam, Cambridge, Mass.) and Catalog #05-1066 (clone El1D7) (Milipore, Billerica, Mass.)) and polyclonal anti-insulin antibodies (e.g., #PA1-36117 (Pierce Biotechnology, Rockford, Ill.). Secondary antibodies conjugated to cyanine (Cy2) or Texas Red (TxR) labels were obtained from Jackson ImmunoResearch, Inc. (West Grove, Pa.) (goat-anti-mouse (Catalog #115-225-146 and #115-075-146) and goat-anti-rabbit (Catalog #111-225-144 and #111-075-144) antibodies).

Preliminary data demonstrated that adult human ONe stem cells are positive for insulin. In addition, preliminary data demonstrated that adult human ONe stem cells express measurable levels of insulin and secrete measurable levels into the medium. Further, preliminary data demonstrated that insulin was produced in a glucose-dependent fashion.

In addition, the Insulin (Human) ELISA kit is used with its companion anti-insulin antibodies (Phoenix Pharmaceuticals, Inc., Burlingame, Calif. (Catalog #EK-035-06)) to quantitatively determine the intracellular levels of insulin and the amount of insulin secreted into the media.

### Example 3

### Insulin Production by Adult Human ONe Stem Cells Under Different Conditions

Experiments are performed to demonstrate that engrafted adult human ONe stem cells produce insulin. Briefly, adult human ONe stem cells are engrafted into a rat pancreas. Insulin-production from adult human ONe stem cells is detected using immunofluorescent staining with an anti-insulin antibody described above and human antinuclear antibodies are used to identify the engrafted cells in the rat pancreas. In addition, the presence and amount of C peptide can be determined using a polyclonal antibody to the C peptide (Bioss, Inc., Woburn, Mass. (Catalog #bs-0274R).

In addition, experiments are performed to determine which insulin receptor(s) (e.g., IRa or IRb) is/are present on the surface of adult human ONe stem cells using immunolocalization. In addition, the levels of mRNA encoding each insulin receptor is evaluated using Northern blotting and RT-PCR.

Further, experiments are performed to determine which factors influence the production of insulin by adult human ONe stem cells in vitro. Briefly, the production of insulin by adult human ONe stem cells can be evaluated in the presence of, for example, glucose or other carbon sources using immunofluorescent methods as described herein.

### Example 4

### In Vivo Engraftment for the Treatment of Diabetes

Experiments are performed to demonstrate that adult human ONe stem cells can be used in an animal model of diabetes (e.g., the NOD mouse or any of the rat models for type I diabetes; see, for example, Mordes et al., 2004, “Rat models of type 1 diabetes: genetics, environment, and autoimmunity,” ILAR 1, 45:278-91) to produce insulin. Adult human ONe stem cells are engrafted into the diabetic animal via direct injection into the pancreas.

Following engraftment, the animals are evaluated weekly, with improvements observed within 6 weeks. Animals can be evaluated biochemically (e.g., measuring plasma and pancreatic levels of insulin), histologically (e.g., detecting the presence of beta cells), and behaviorally (e.g., general health, activity, food consumption, weight gain) for improvement relative to non engrafted control animals.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of treating a patient having diabetes, comprising:
culturing human olfactory neuroepithelium stem cells ex vivo under appropriate conditions; and
engrafting the human olfactory neuroepithelium stem cells into the patient, wherein the human olfactory neuroepithelium stem cells produce insulin, thereby treating the patient having diabetes.

2. A method of treating a patient having diabetes, comprising:
providing human olfactory neuroepithelium stem cells that have been cultured under appropriate conditions ex vivo; and
engrafting the cultured human olfactory neuroepithelium stem cells into the patient, wherein the human olfactory neuroepithelium stem cells produce insulin, thereby treating the patient having diabetes.

3. A method of treating a patient having diabetes, comprising:
harvesting human olfactory neuroepithelium stem cells from the patient having diabetes;
culturing the human olfactory neuroepithelium stem cells ex vivo under appropriate conditions; and
engrafting the human olfactory neuroepithelium stem cells into the patient, wherein the human olfactory neuroepithelium stem cells produce insulin, thereby treating the patient having diabetes.

4. The method of any of the preceding claims, wherein the human olfactory neuroepithelium stem cells are autologous to the patient.

5. The method of any of the preceding claims, wherein the human olfactory neuroepithelium stem cells are allogeneic to the patient.

6. The method of any of the preceding claims, wherein the harvesting step is performed using an endoscope.

7. The method of any of the preceding claims, wherein the culturing step increases the number of human olfactory neuroepithelium stem cells.

8. The method of any of the preceding claims, wherein the human olfactory neuroepithelium stem cells are engrafted into the pancreas of the patient.

9. The method of any of the preceding claims, wherein the engrafting step comprises injecting the human olfactory neuroepithelium stem cells into the pancreas.

10. The method of any of the preceding claims, wherein the culturing step comprises adjusting the amount of glucose in the medium.
